(19) United States
(12) Patent Application Publication
(10) Pub. No.: US 2026/0248979 A1
(43) Pub. Date: Aug. 27, 2026

(54) PURIFICATION METHOD AND PURIFICATION SYSTEM FOR AIRBORNE MOLECULAR CONTAMINANTS

(71) Applicant: GREENFILTEC LTD., Taoyuan City (TW)

(72) Inventors: Ju-Ting LEE, Taoyuan City (TW); Yu-De LIEN, Taoyuan City (TW); Ching-Han LIU, Taoyuan City (TW); Ching-Chuan SU, Taoyuan City (TW)

(21) Appl. No.: 19/289,135

(22) Filed: Aug. 4, 2025

(30) Foreign Application Priority Data
Feb. 21, 2025 (TW) 114106571

(51) Int. Cl. A61L 9/20; A61L 9/14

(52) U.S. Cl. CPC A61L 9/205; A61L 9/14

(57) ABSTRACT

Provided are a purification method and a purification system for AMC. The purification method comprises steps of: mixing AMC to be treated with a first water mist to obtain a gas-liquid mixture; subjecting the gas-liquid mixture to contact photocatalysts on a porous substrate under radiation to react into a first treated mixture; washing the first treated mixture and the porous substrate with a second water mist to obtain a water washed solution; and discharging a purified gas and the water washed solution. The molecular weight of the first treated mixture is lower than that of the AMC to be treated, and the AMC content in the purified gas is lower than that of the AMC to be treated. The technical means can improve the purification rate of AMC and extend the lifespan of the photocatalysts, thereby further improving overall purification performance.

### CROSS-REFERENCE TO RELATED APPLICATION

Pursuant to 35 U.S.C. § 119 (a), this application claims the benefit of the priority to Taiwan Patent Application No. 114106571 filed Feb. 21, 2025. The content of the prior application is incorporated herein by its entirety.

### BACKGROUND OF THE INVENTION

### 1. Field of the Invention

The present invention relates to gas purification technology, especially for a purification method and a purification system for airborne molecular contaminants.

### 2. Description of the Prior Arts

According to the Standard F21-1102 published by Semiconductor Equipment and Materials International (SEMI), airborne molecular contaminants (AMC) are classified into four types, including acids (MA), bases (MB), condensable organic compounds (MC) and dopants (MD). In addition, AMC can also be classified into acidic gases, basic gases and volatile organic compounds (VOCs). Possible sources include exhaust emissions from automobiles and motorcycles, environmental ozone, exhaust emissions from factories, volatiles of chemical solvents and volatile sour gases from etching.

During the semiconductor production process, the organic solvent evaporated from photo-etchant coating generates AMC in the air. If unable to be removed or purified effectively, AMC tends to settle on the surface of wafers and cause defects, thereby decreasing the yield of products. In addition, accidental leakage of AMC causes individual safety damage and environmental pollution problems. Therefore, semiconductor manufacturers all actively build up a highly clean and isolated cleanroom. To meet cleanroom specifications, a make-up air unit (MAU) is set up at the air inlet to condition the outside air. Alternatively, a fan filter unit (FFU) is installed on the ceiling, so that the airflow is driven by a fan to pass through the high efficiency particulate air filter (HEPA) or ultra-low penetration air filter (ULPA) before entering the cleanroom, so as to purify the air outside or in the cleanroom.

In the past, the activated carbon filter was used for absorption of AMC. However, when activated carbons reach the saturated state, it is necessary to replace the activated carbon filter for further air purification, resulting in a significant increase of the activated carbon filter consumptions and decline of the purification efficiency. Although, photocatalysts can also be used to decompose AMC directly under light exposure, it takes a long period to directly decompose AMC, making it difficult to process a large number of AMC rapidly and efficiently. Furthermore, the product from AMC decomposition will be adsorbed on the photocatalyst surface, making the decomposition efficiency decreased after a period of processing. Therefore, the purification process needs to be interrupted to regenerate the photocatalyst, so that the regenerated photocatalyst could be used for further air purification.

In view of this, the purification technologies above are difficult to meet the current emphasis for enterprises on the environment, social, and governance (ESG), and more difficult to accomplish the expectation of sustainable development goals (SDGs). Therefore, an improvement of the AMC treatment is still in need to overcome the deficiency of the traditional technical means of purification.

### SUMMARY OF THE INVENTION

One objective of the present invention is to improve a purification rate for the AMC and enhance an overall purification performance.

Another objective is to prolong lifetime of the photocatalysts for purification of the AMC and to improve the problem of frequent interruption during a purification process to regenerate the photocatalysts.

To achieve the foresaid objectives, the present invention provides the purification method for the AMC, the purification method comprising:
mixing an AMC to be treated and a first water mist to obtain a gas-liquid mixture;
subjecting the gas-liquid mixture to react with a photocatalyst on a porous substrate under a radiation source, so as to react the AMC to be treated into a first treated composition;
washing the first treated composition and the porous substrate with a second water mist to obtain a water washed solution, wherein the first treated composition is dissolved in the water washed solution; and
exhausting a purified gas and the water washed solution, wherein a molecular weight of the first treated composition is lower than a molecular weight of the AMC to be treated, and a content of the AMC in the purified gas is lower than a content of the AMC to be treated.

By mixing the AMC to be treated with the first water mist before it reacts with the photocatalysts on the porous substrate under the radiation source for decomposition into the first treated composition, it is beneficial to shortening the treatment time required and improving the purification rate for the AMC to be treated. In addition, washing the porous substrate with the second water mist helps dissolve the first treated composition on the porous substrate into the water washed solution and simultaneously remove it from the surface of the photocatalysts during the purification process, achieving the photocatalysts regeneration. Therefore, the technical means of the present invention can prolong a lifetime of the photocatalysts and solve the problems of the frequent interruption during the purification process for photocatalysts regeneration. Particularly, the purification method of the present invention enhances the overall purification performance and reduces the AMC content in the purified gas to a lower level. Overall, the purification method for the AMC can overcome the deficiencies in the past, and it is more conducive for companies to achieving the ESG and SDGs goals.

On the other hand, the AMC to be treated in the gas-liquid mixture can be decomposed into the first treated composition with a smaller molecular weight (such as an acid, a base or an organic compound) upon contact with the photocatalysts, resulting in the molecular weight of the first treated composition being lower than that of the AMC to be treated. After the AMC is decomposed into the first treated composition by the photocatalyst, a solubility of the first treated composition in the second water mist is larger than that of the AMC in the second water mist. Consequently, the first treated composition produced through photocatalytic decomposition is more soluble in the water washed solution and is discharged more efficiently, resulting in a lower AMC content in the purified gas compared to the AMC to be treated.

Optionally, the AMC to be treated contains acetone, isopropanol, toluene or any combinations thereof, but is not limited thereto. In one of the embodiments, the AMC to be treated may be substantially free of a polyhalogenated compound, a benzenoid compound, a polycyclic aromatic hydrocarbon, a long-chain alkane or any combinations thereof. Take the AMC to be treated containing acetone as an example: the acetone in contact with the photocatalysts can be decomposed into a formic acid (the first treated composition), such that the molecular weight of the first treated composition is lower than that of the AMC to be treated, and the solubility of the first treated composition in the second water mist is larger than that of the acetone in the second water mist. Accordingly, the AMC can be decomposed into the first treated composition with higher solubility and smaller molecular weight, and the first treated composition can be dissolved in the water washed solution and also discharged by water washing.

In the purification method, the AMC to be treated may be fed into a chamber with an inlet flow rate, and mixed with the first treated composition in the chamber to obtain the gas-liquid mixture. Optionally, an inlet quantity of the AMC to be treated may be 20 m³/hr to 5000 m³/hr, but is not limited thereto. Optionally, the inlet flow rate of the AMC to be treated may be 0.1 m/min to 2.5 m/min, but is not limited thereto. In the present specification, the inlet flow rate (also called face velocity) is defined as the flow rate of the AMC to be treated flowing through a section of the chamber. Under a condition of the same inlet quantity, the larger the area of the section, the smaller the inlet flow rate, in contrary, the smaller the area of the section, the larger the inlet flow rate.

In the purification method, a composition of the second water mist contains water, a mixture of water and an organic solvent, a mixture of water and an inorganic solvent or any combinations thereof, wherein a water proportion of the second water mist is 80% of a volume percentage or more, or 80% to 100% of the volume percentage. Optionally, a flow rate of the first water mist and a flow rate of the second water mist are each 0.1 L/min to 1 L/min and 1.3 L/min to 1 L/min respectively, but not limited thereto. For examples, the composition of the second water mist may be water, a methanol, an ethanol, an acetic acid or any combinations thereof. A person skilled in the art can choose the suitable second water mist to wash the first treated composition and the porous substrate according to different needs. In one of the embodiments, the first water mist is equal to the second water mist, that is, the first water mist and the second water mist have a same water source. The first water mist may be mixed with the AMC to be treated to obtain the gas-liquid mixture, and also washes the first treated composition and the porous substrate, so that the first water mist plays the role of gas-liquid mixing and photocatalyst regeneration. When the first water mist is same as the second water mist, the flow rate of the first water mist is 0.1 L/min to 1 L/min.

Preferably, the purification method further comprises:
recycling the water washed solution;
separating the first treated composition from the water washed solution to obtain a regeneration solution;
spraying the regeneration solution to produce a regeneration water mist; and
mixing the regeneration water mist and the AMC to be treated to obtain the gas-liquid mixture, and washing the first treated composition and the porous substrate with a combination of the regeneration water mist and the second water mist.

Accordingly, the first treated composition can be separated from the water washed solution through filtration to recycle the water washed solution into the reusable regeneration solution, so that the purification method can reduce the water waste (namely reducing the usage amount of the first water mist and/or the second water mist) and an amount of a discharged water, further meeting expectations such as green purification, water recycling and environmental protection as well as the ESG and SDGs goals.

Optionally, a composition of the regeneration water mist contains water, a mixture of water and an organic solvent, a mixture of water and an inorganic solvent or any combinations thereof. A water proportion of the regeneration water mist is 80% of a volume percentage or more, or 80% to 100% of the volume percentage.

In addition, to achieve the foresaid objectives, the present invention provides a purification system for the AMC, the purification system comprising:
a chamber, having a channel formed with an air inlet and an air outlet connected to each other;
a first gas treatment module located between the air inlet and the air outlet, and the first gas treatment module including at least one treatment unit, each treatment unit including:
   a housing having a windward panel and a side panel, the side panel positioned around the windward panel to form a treatment space, the windward panel formed with at least one inlet hole, and the side panel formed with at least one outlet hole;
   at least one porous substrate with photocatalysts disposed in the treatment space;
   at least one radiation source facing to the at least one porous substrate with photocatalysts; and
a water mist production module including an inlet pipe and a water mist sprayer, the water mist sprayer installed on the windward panel, wherein the water mist sprayer include a spray nozzle facing toward the treatment space.

With the above design, the AMC to be treated fed into the chamber from the air inlet must flow through the first gas treatment module, and mix with the first water mist of the water mist production module in the treatment space of the at least one treatment unit, then the gas-liquid mixture contacts the photocatalyst in the treatment space to decompose into the first treated composition, so as to shorten the time needed for treating the AMC and improve the purification rate for the AMC to be treated. In addition, washing the porous substrate with the second water mist is more conducive to dissolve the first treated composition on the porous substrate in the water washed solution, and washes the first treated composition away from the surface of the photocatalyst for photocatalysts regeneration, so that the present invention is more conducive to the lifetime prolongation of the photocatalysts to solve the problem of the frequent interruption during the purification process to regenerate the photocatalyst. In particular, the purification system for the AMC also improves the overall purification performance, and has many advantages compared with conventional purification devices.

In the purification system, the windward panel has a bearing hole installed with the water mist sprayer, and the number of the at least one inlet hole is plural. Accordingly, the AMC to be treated fed through the air inlet is evenly dispersed by the multiple inlet holes and enters the treatment space to carry out gas-liquid mixing with the first water mist, and contacts the photocatalyst to be decomposed into the first treated composition, then the purified gas is evenly dispersed by the outlet holes and exhausted from the treatment space.

In the purification system, the inlet holes are formed along an airflow direction, the outlet holes are formed along a diffuser direction, and the diffuser direction is non-parallel to the airflow direction. Optionally, the airflow direction intersects with the diffuser direction to subject the AMC to be treated to enter the treatment space through the air inlet, and exhausts the treatment space through the air outlet after reacting with the photocatalyst, so as to extend the time of the AMC to be treated staying in the treatment space and contacting the photocatalyst.

In the purification system, the number of the at least one porous substrate is plural, comprising a first porous substrate with photocatalysts and a second porous substrate with photocatalysts located in the treatment space and opposite to each other; the number of at least one radiation source is plural, parts of the radiation sources are spaced apart from each other and face to the first porous substrate with photocatalysts, and other parts of the radiation sources are spaced apart from each other and face to the second porous substrate with photocatalysts. For example, two to four of the radiation sources are spaced apart from each other and disposed next to the first porous substrate with photocatalysts, and other two to four of the radiation sources are spaced apart from each other and disposed next to the second porous substrate with photocatalysts, so that the radiation sources are evenly irradiated onto the photocatalysts of the first and second porous substrates, allowing the AMC to be decomposed by photocatalyst under the radiation irradiation and turned into the first treated composition with the lower molecular weight and the higher solubility in the second water mist under.

Optionally, the first porous substrate with photocatalysts is positioned diagonally to the second porous substrate with photocatalysts in the treatment space, and a distance between one side of the first porous substrate with photocatalysts and one side of the second porous substrate with photocatalysts that are close to the windward panel is larger than a distance between the other side of the first porous substrate with photocatalysts and the other side of the second porous substrate with photocatalysts that are far from the windward panel, that is, the first porous substrates with photocatalysts and the second porous substrates with photocatalysts are positioned in a V-shaped arrangement. Compared with a parallel arrangement, the above V-shaped arrangement makes the porous substrates have the larger surface in the treatment space, so as to attach more of the photocatalysts on the porous substrate. Optionally, the radiation sources contain multiple ultraviolet lamps, an angle between one of the ultraviolet lamps facing to the first porous substrate with photocatalysts and another one of the ultraviolet lamps facing to the second porous substrate with photocatalysts may be 30° to 60°. That is, two of the ultraviolet lamps located next to the first porous substrate with photocatalysts and the second porous substrate with photocatalysts, and opposite to each other may be positioned in the V-shaped arrangement, corresponding to the arrangement of the first and porous substrates with photocatalysts.

Optionally, the number of the ultraviolet lamps facing to the first porous substrate with photocatalysts is equal to the number of the ultraviolet lamps facing to the second porous substrate with photocatalysts and located opposite to each other in the treatment space. In addition, a height of the ultraviolet lamps facing to the first porous substrate with photocatalysts is approximately equal to a height of the ultraviolet lamps facing to the second porous substrate with photocatalysts.

Optionally, the parts of the radiation sources are positioned between the first porous substrate with photocatalysts and the side panel, and the other parts of the radiation sources are positioned between the second porous substrate with photocatalysts and the side panel.

In the purification system, a material of the at least one porous substrate is selected from the group consisting of foamed ceramic, activated carbon, metal organic framework, foamed metal and any combinations thereof. The porous structure design of the porous substrates can increase the specific surface area of the porous substrates, thereby increasing the number of the photocatalysts attached to the porous substrate. Also, the porous structure design enhances a diffuser effect and extends the time of the AMC staying in the treatment space and contacting the photocatalyst, so as to improve the overall purification performance Optionally, the specific surface area of the porous substrates is 1000 m²/g to 3500 m²/g.

Optionally, the porous substrates have a pore size of 50 mesh to 200 mesh. It can be understood that the mesh in the present specification represents a number of the mesh of the porous substrates in an area of 1 inch×1 inch, the larger the mesh, the more the pores.

Optionally, a thickness of the porous substrates may be 10 mm to 100 mm, or 10 mm to 60 mm without particular limitations as long as the radiation sources can reach the photocatalyst on the porous substrates to decompose the AMC.

Optionally, the radiation source may be selected from the group consisting of an ultraviolet source, an infrared source, a microwave source, a plasma source, an X-ray source, an ion beam source, and any combinations thereof. When the radiation source is the ultraviolet source, a power of the ultraviolet source may be 10 W to 1000 W, and a wavelength of the ultraviolet source may be 245 nm to 365 nm.

Preferably, to further improve the overall purification performance, the number of the at least one treatment unit of the first gas treatment module is plural. Optionally, the treatment units of the first gas treatment module are arranged in a matrix.

Preferably, the windward panels of the treatment units are arranged closely and extend to an inner wall of the channel, so that the AMC to be treated fed from the air inlet can be purified by the treatment units and converted into the purified gas.

To improve the overall purification performance, the purification system also comprises a second gas treatment module, the first gas treatment module and the second gas treatment module are positioned between the air inlet and the air outlet and spaced from each other; the second gas treatment module comprises at least one treatment unit, and the at least one treatment unit of the first gas treatment module is identical to the at least one treatment unit of the second gas treatment module. For example, the number of the at least one treatment unit of the second gas treatment module is plural, the windward panel of the treatment units of the second gas treatment module arranged closely and extending to an inner wall of the channel.

Preferably, multiple treatment units are detachably assembled with each other and form the first gas treatment module. Likewise, multiple treatment units are detachably assembled with each other and form the second gas treatment module. Optionally, a person skilled in the art can assemble the first and second gas treatment modules by welding, riveting or locking the treatment units, but not limited thereto. A person skilled in the art can also weld, rivet or lock the treatment units onto a frame to assemble the first gas treatment module and the second gas treatment module optionally. Through the detachable design above, the specific single treatment unit can be detached from the purification system during the purification process as required, instead of changing the whole gas treatment module. Therefore, the design of the purification system not only simplifies the maintenance and reduces the repair cost, but also declines the production of wasted treatment units, so that the purification technology meets many expectations such as green purification, waste reduction and environmental protection. It is more beneficial for implementation of ESG, and more conducive to achieve the ultimate goals of net zero emission and sustainable development.

In the purification system, it may further comprise the at least one water collection tank disposed under the first gas treatment module and/or the second gas treatment module.

In the purification system, it also comprises a return pipe connected with the at least one water collection tanks and the inlet pipe, and a filtration unit is installed on the return pipe and branched with a discharge pipe. Accordingly, the water washed solution collected from the water collection tanks can be transported into the filtration unit through the return pipe, so that the water washed solution can be separated into the first treated composition and the regeneration solution by the filtration unit. The reusable regeneration solution flows through the inlet pipe into the treatment space, and the first treated composition is discharged through the discharge pipe. Accordingly, the purification system has the advantages such as reducing the water waste and declining the amount of the discharged water. It is more conducive to meet the expectations of the green purification process, the recycle of the water source and the environmental protection, and is beneficial to implementation the ESG and achieve the ultimate goals of the sustainable development.

### BRIEF DESCRIPTION OF THE DRAWINGS

FIG. 1 is a flowchart of a first purification method for AMC.

FIG. 2 is a flowchart of a second purification method for AMC.

FIG. 3 is a perspective view of a first embodiment of a purification system for AMC.

FIG. 4 and FIG. 5 are exploded views of the gas treatment unit from different angles.

FIG. 6 is a top view of the gas treatment unit.

FIG. 7 is a side view of the gas treatment unit of the first gas treatment module connected to the water mist production module.

FIG. 8 is a front view of a second embodiment of a purification system for AMC.

FIG. 9 is a side view of the second embodiment of the purification system for AMC.

FIG. 10 is a perspective view of a third embodiment of a purification system for AMC.

FIG. 11 is a side view of the third embodiment of the purification system for AMC.

FIG. 12 is a schematic view of the gas treatment unit connected to a return pipe and a filtration unit.

FIG. 13 is a graph showing the removal efficiencies of isopropanol, acetone, and toluene over time during the continuous purification of AMC in Example 1.

FIG. 14 is a graph showing the acetone removal efficiencies of AMC to be treated after 1 hour of purification in Example 1, Comparative Examples 1 and 2, and Control Example.

FIG. 15 is a graph showing the acetone removal efficiencies of AMC to be treated after 1 hour of purification in Example 1 and Comparative Example 3.

FIG. 16 is a graph showing the acetone removal efficiency of AMC to be treated in Example 2 measured at different times during continuous purification processes.

### DETAILED DESCRIPTION OF THE PREFERRED EMBODIMENTS

Hereinafter, the implementation of the purification method and the purification system for AMC are illustrated below with figures. Comparative examples and control examples are also described below. A person skilled in the art can easily realize the advantages and effects of the present invention from the following examples, comparative examples, and control examples. Various modifications and variations could be made in order to practice or apply the present invention without departing from the spirit and scope of the present application.

### Purification Method for AMC

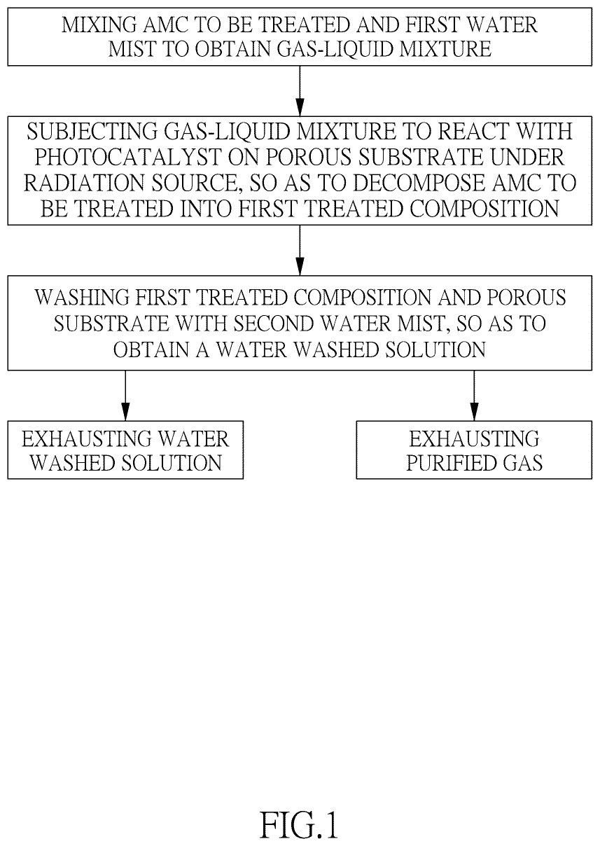

According to FIG. 1, the first purification method for the AMC comprises:
mixing an AMC to be treated and a first water mist to obtain a gas-liquid mixture;
subjecting the gas-liquid mixture to react with a photocatalyst on a porous substrate under a radiation source, so as to decompose the AMC to be treated into a first treated composition;
washing the first treated composition and the porous substrate with a second water mist to obtain a water washed solution, wherein the first treated composition is dissolved in the water washed solution; and
exhausting a purified gas and the water washed solution, wherein a molecular weight of the first treated composition is lower than a molecular weight of the AMC to be treated, and a content of the AMC in the purified gas is lower than a content of the AMC to be treated.

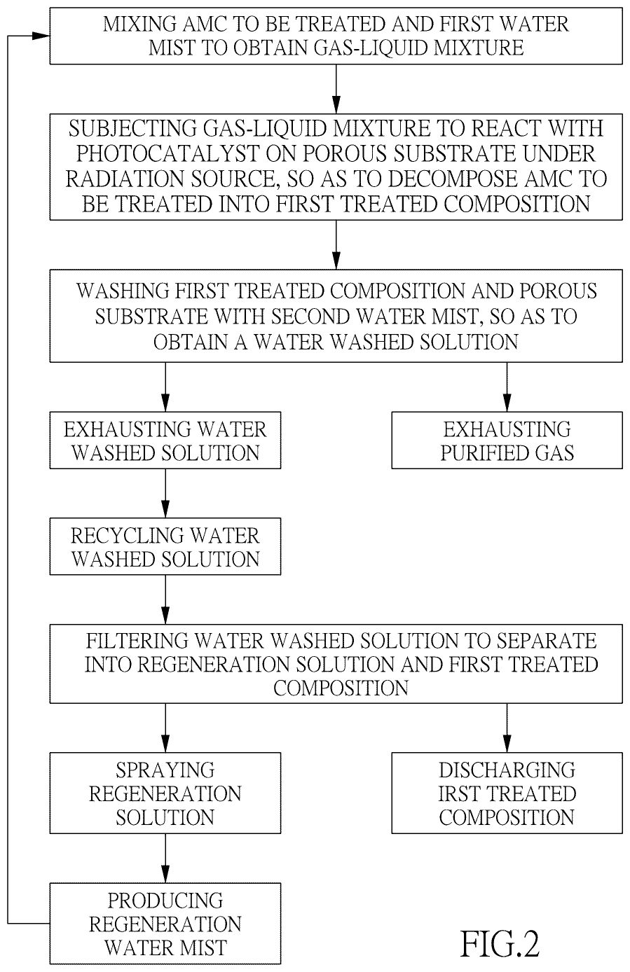

According to FIG. 2, a second purification method for the AMC comprises:
mixing the AMC to be treated and the first water mist to obtain the gas-liquid mixture;
subjecting the gas-liquid mixture to react with a photocatalyst on the porous substrate under the radiation source, so as to decompose the AMC to be treated to the first treated composition;
washing the first treated composition and the porous substrate with the second water mist to obtain the water washed solution, wherein the first treated composition is dissolved in the water washed solution;
exhausting the purified gas and the water washed solution, wherein a molecular weight of the first treated composition is lower than a molecular weight of the AMC to be treated, and a content of the AMC in the purified gas is lower than a content of the AMC to be treated; and
recycling the water washed solution;
filtering the water washed solution to separate a regeneration solution and the first treated composition from the water washed solution;
spraying the regeneration solution to produce a regeneration water mist; and
mixing the regeneration water mist and the AMC to be treated to obtain the gas-liquid mixture, and washing the first treated composition and the porous substrate with a combination of the regeneration water mist and the second water mist.

### Purification System for AMC

In the first embodiment of the purification system, only one single treatment unit is installed as a first gas treatment module to conduct an AMC purification.

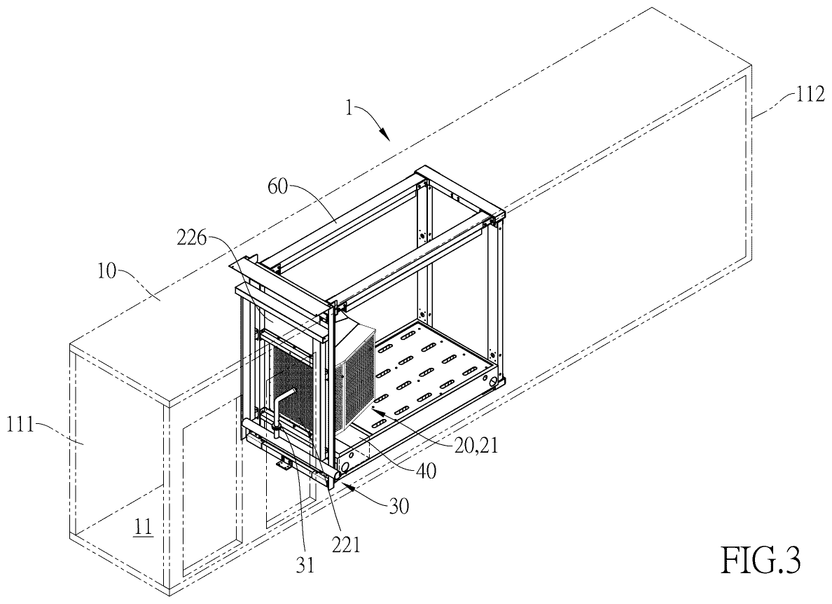

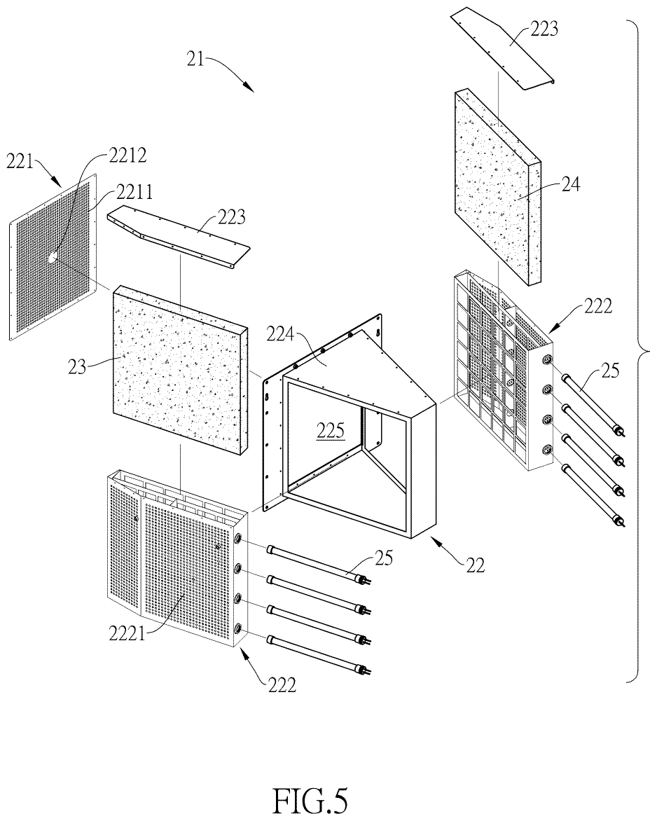

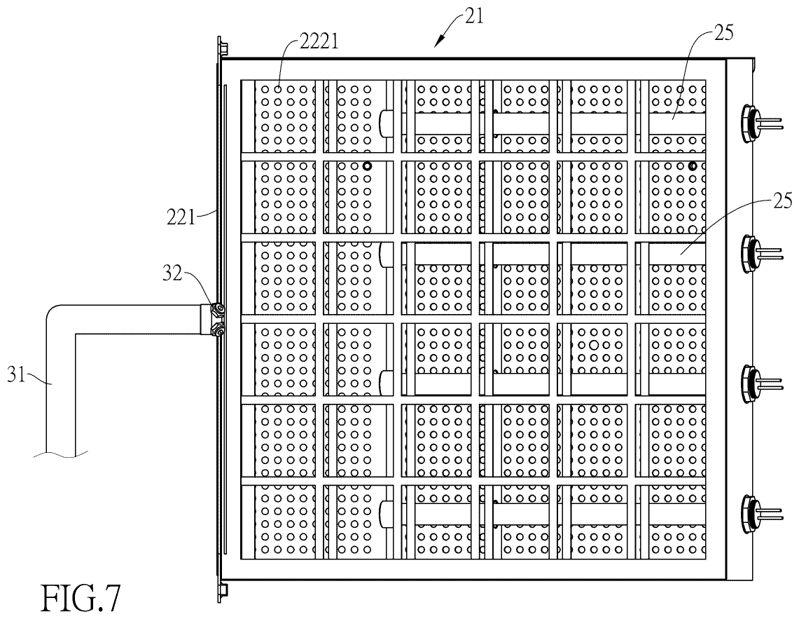

Referring to FIGS. 3 to 7, the purification system 1 for AMC includes a chamber 10, a first gas treatment module 20, a water mist production module 30 and a water collection tank 40.

The chamber 10 has a channel 11 formed with an air inlet 111 and an air outlet 112 connected to each other, facilitating feeding the AMC to be treated into the chamber 10. After purifying by the first gas treatment module 20, the produced purified gas can be exhausted to the outside of the chamber 10 through the air outlet 112.

The first gas treatment module 20 is positioned between the air inlet 111 and the air outlet 112, a single treatment unit 21 of the first gas treatment module 20 includes a housing 22, a first porous substrate with photocatalysts 23, a second porous substrate with photocatalysts 24 and multiple ultraviolet lamps 25.

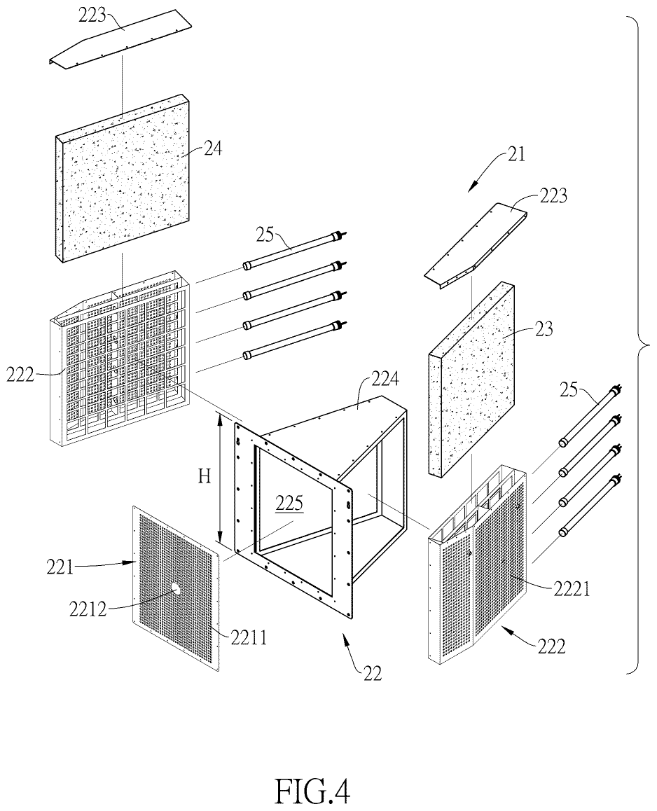

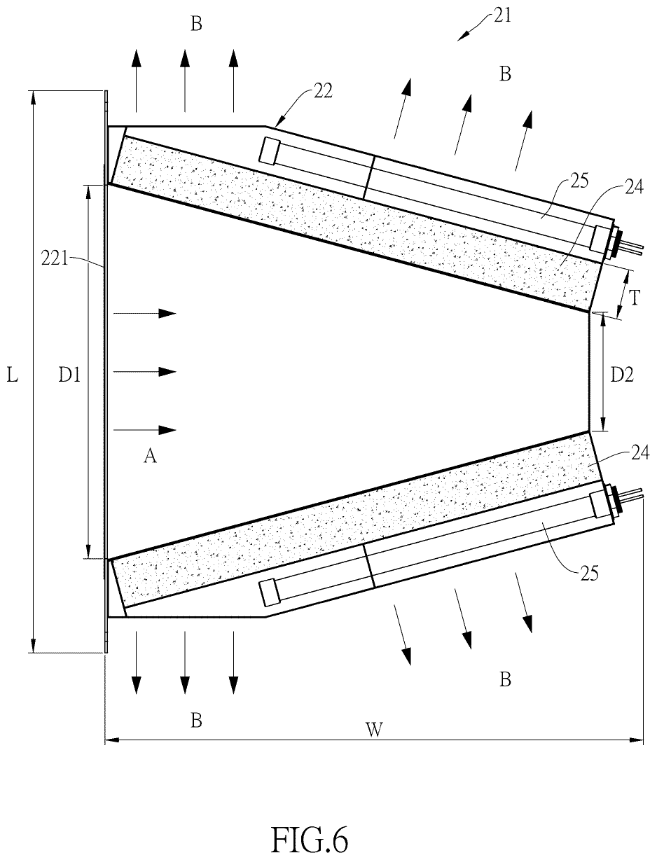

As shown in FIG. 4 to FIG. 6, the housing 22 has a windward panel 221, two side panels 222, two cover plates 223 and a support frame 224. The side panels 222 and the cover plates 223 connect to the support frame 224, and the side panels 222, the cover plates 223 and the support frame 224 are disposed around the windward panel 221 together to form a treatment space 225. The windward panel 221 is formed with a bearing hole 2212 and multiple inlet holes 2211 formed around the bearing hole 2212, and the side panels 222 are each formed with multiple outlet holes 2221. As shown in FIG. 6, the inlet holes 2211 are formed along an airflow direction A defined by the air inlet 111 and the air outlet 112, and the outlet holes 2221 are formed along a diffuser direction B, which is non-parallel to the airflow direction A.

The first porous substrate with photocatalysts 23 and the second porous substrate with photocatalysts 24 are located in the treatment space 225 and opposite to each other. In particular, the first porous substrate with photocatalysts 23 and the second porous substrate with photocatalysts 24 are positioned adjacent to one of the two side panels 222 formed with the outlet holes 2221. The first porous substrate with photocatalysts 23 is positioned diagonally to the second porous substrate with photocatalysts 24, and a distance D1 between one side of the first porous substrate with photocatalysts 23 and one side of the second porous substrate with photocatalysts 24 that are close to the windward panel 221 is larger than a distance D2 between the other side of the first porous substrate with photocatalysts 23 and the other side of the second porous substrate with photocatalysts 24 that are far from the windward panel 221. In other words, the first porous substrates with photocatalysts 23 and the second porous substrates with photocatalysts 24 are positioned in a V-shaped arrangement to enlarge the surfaces of the first porous substrates with photocatalysts 23 and the second porous substrates with photocatalysts 24.

The multiple ultraviolet lamps 25 are positioned facing the first porous substrates with photocatalysts 23 and the second porous substrate with photocatalysts 24. Each of the two side panels 222 is installed with four ultraviolet lamps 25, the four ultraviolet lamps 25 are spaced apart from each other and disposed between the first porous substrates with photocatalysts 23 and one of the side panels 222, and another four ultraviolet lamps 25 are spaced apart from each other and disposed between the second porous substrates with photocatalysts 24 and the other of the side panels 222.

As shown in FIG. 6, an angle between one ultraviolet lamp 25 facing the first porous substrate with photocatalysts 23 and another ultraviolet lamp 25 facing the second porous substrate with photocatalysts 24 is 30° to 60°. That is, two of the ultraviolet lamps 25 are positioned in a V-shaped arrangement.

The water mist production module 30 is connected to the first gas treatment module 20. The water mist production module 30 includes an inlet pipe 31 and a water mist sprayer 32 connected to each other, the water mist sprayer 32 is installed on the bearing hole 2212 of the windward panel 221, and a spray nozzle of the water mist sprayer 32 faces the treatment space 225, such that the first water mist is fed into the first treatment 225 and mixed with the AMC to be treated for gas-liquid mixing.

As shown in FIG. 4 and FIG. 6, a length L, a width W, and a height H of each treatment unit 21 are adjustable as required without particular limitations. In one of the embodiments, the porous substrate has a specific surface area of 1000 m²/g to 3500 m²/g, a pore size of 50 mesh to 200 mesh, and a thickness T (as shown in FIG. 6) of 50 mm to 60 mm.

The water collection tank 40 is disposed under the first gas treatment module 20 to collect the water washed solution overflowing from the first gas treatment module 20. The first gas treatment module 20 may be installed on a metal frame 60 including a wind screening plate 226, and the foresaid windward panel 221, the metal frame 60, the wind screening plate 226 are arranged closely and extending to an inner wall of the channel 11.

With above structural configuration, the AMC to be treated fed into the chamber 10 from the air inlet 111 inevitably flows through the treatment unit 21 of the first gas treatment module 20, and contacts the photocatalyst after mixing with the first water mist from the water mist sprayer 32 in the treatment space 225, such that the AMC contained in the gas to be treated can be decomposed into the first treated composition more effectively. The spraying range of the water mist sprayers 32 can cover the locations of the first porous substrates with photocatalysts 23 and the second porous substrates with photocatalysts 24, so that the first treated composition on the porous substrate dissolvable in the water washed solution can be washed away from the surface of the photocatalyst at the same time for photocatalysts regeneration, which is beneficial to prolong lifetime of the photocatalysts and solve the problem of frequent interruption during the purification process to regenerate the photocatalysts. The purification system is effective to improve the purification performance and decrease the AMC content in the purified air.

In the second embodiment of the purification system, the first gas treatment module includes multiple treatment units, which are substantially the same as described in the first embodiment.

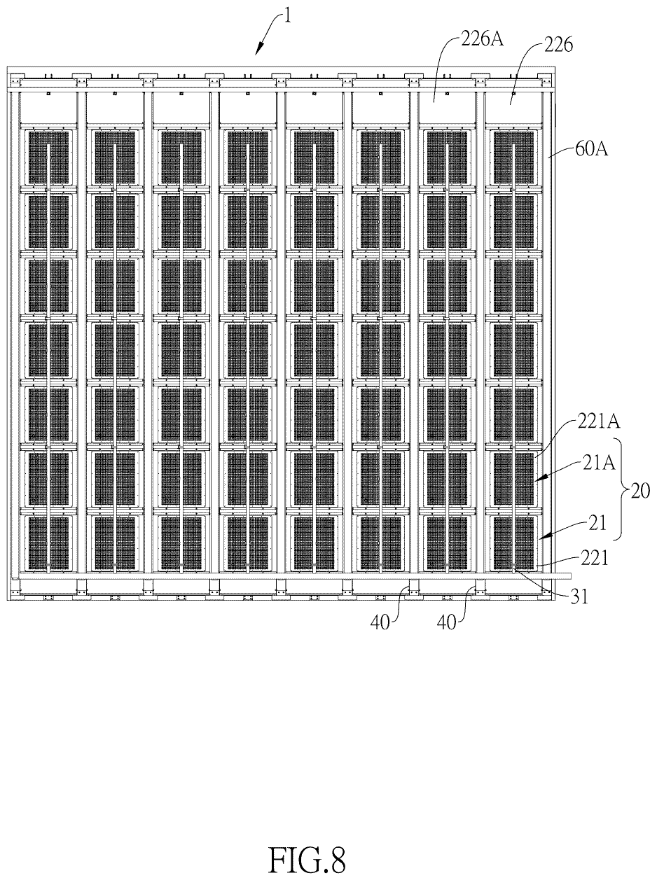

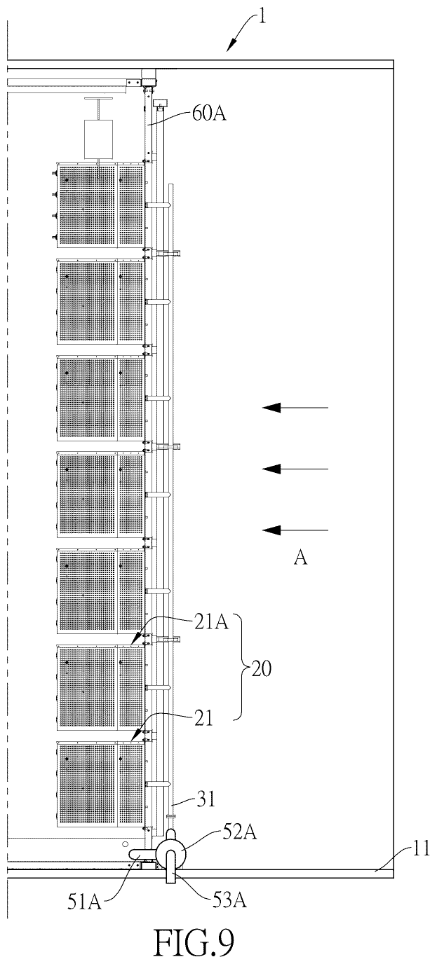

Referring to FIG. 8 and FIG. 9, the purification system for the AMC of the second embodiment includes a first gas treatment module 20 as described in the first embodiment, and the first gas treatment module 20 includes multiple treatment units 21, 21A. A frame 60A can be used to carry the multiple treatment units 21, 21A, detachably positioned on the frame 60A and arranged in a matrix. Multiple wind screening plates 226, 226A are disposed on the frame 60A. The windward panels 221,221A, the frame 60A, and the wind screening plates 226, 226A are arranged closely and extend to the inner wall of the channel 11, so that the AMC to be treated must flow through the multiple treatment units 21, 21A of the first gas treatment module 20 along the airflow direction A illustrated in FIG. 9, and mix with the water mist in the treatment space then contact the photocatalyst to increase an overall purification capacity through the multiple treatment units 21, 21A.

As described in the first embodiment, the purification system 1 for the AMC of the second embodiment also has the water collection tank 40 located under the first gas treatment module 20. Different from the first embodiment, the purification system 1 for the AMC has a return pipe 51A connected with the water collection tank 40 and the inlet pipe 31, a filtration unit 52A installed on the return pipe 51A and branched with a discharge pipe 53A. Accordingly, the water washed solution collected from the water collection tank 40 can be separated into the regeneration solution and the first treated composition by the filtration unit 52A, the first treated composition may be discharged through the discharge pipe 53A, and the regeneration solution may be returned via the inlet pipe 31 with a driving device (not shown in the figure, such as a motor) installed on the return pipe 51A for recycling and reusing in the purification system, then continuously sprayed into the regeneration water mist for the gas-liquid mixing and the water washing steps as described above.

In the third embodiment of the present invention of the purification system, the purification system includes multiple gas treatment modules, and each of the gas treatment modules includes multiple treatment units, which are substantially the same as the first embodiment.

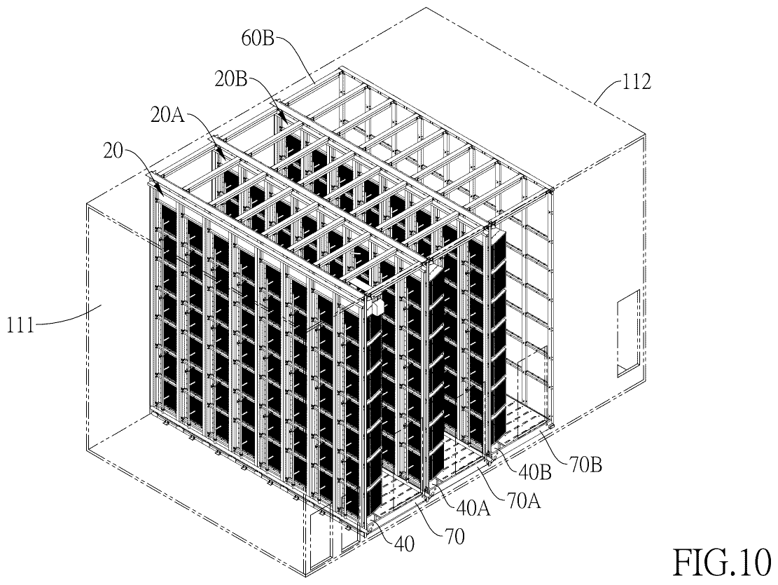

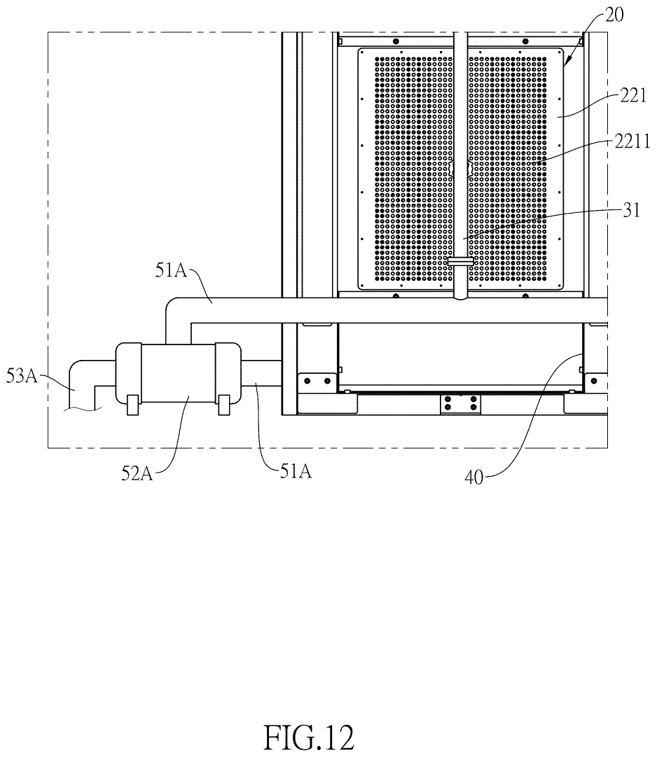

Referring to FIG. 10 and FIG. 12, the purification system 1 for the AMC of the third embodiment includes the first gas treatment module 20 as described in the first embodiment, a similar second gas treatment module 20A, and a third gas treatment module 20B. A frame 60B is placed to install the first gas treatment module 20, the second gas treatment module 20A, and the third gas treatment module 20B, which are spaced apart from each other and disposed between the air inlet 111 and the air outlet 112. Besides, the water collection tanks 40, 40A, 40B are disposed under the first gas treatment module 20, the second gas treatment module 20A, and the third gas treatment module 20B, respectively. As shown in FIG. 10, an access door 70 is disposed between the first gas treatment module 20 and the second gas treatment module 20A, another access door 70A is disposed between the second gas treatment module 20A and the third gas treatment module 20B, and further another access door 70B is disposed between the third gas treatment module 20B and the air outlet 112. Therefore, the specific treatment units can be replaced as required through the access doors 70, 70A, 70B.

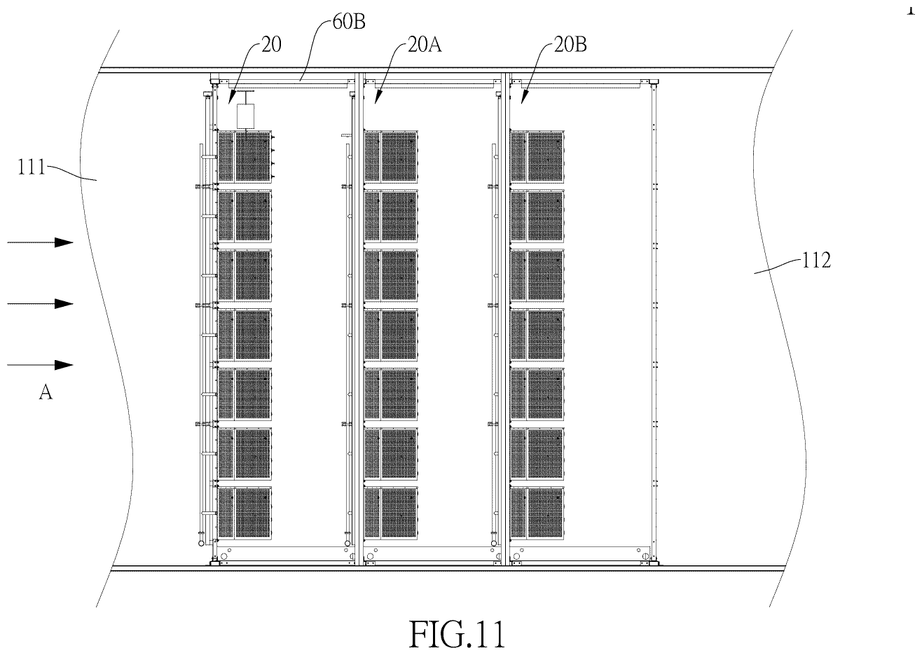

As shown in FIG. 10 and FIG. 11, each of the gas treatment modules includes multiple treatment units arranged in a matrix, and the treatment units are substantially the same or different. With the installation of multiple gas treatment modules, the AMC to be treated can sequentially flow through the first gas treatment module 20, the second gas treatment module 20A, and the third gas treatment module along the airflow direction A as shown in FIG. 11, so as to perform multiple repeated purification processes in sequence to enhance the overall purification performance.

As shown in FIG. 12, the water washed solution collected from the water collection tank 40 was sent into the filtration unit 52A through the return pipe 51A to separate the water washed solution into the regeneration solution and the first treated composition. The first treated composition may be discharged by the discharge pipe 53A, and the regeneration solution may be returned via the inlet pipe 31 by a driving device (not shown in the FIG. installed thereon to allow the recycle and reuse in the purification system, then continuously sprayed into the regeneration water mist for the gas-liquid mixing and the water washing steps.

### Example 1

To verify the beneficial effects of the purification method and the purification system for the AMC of the present invention, a purification system as shown in FIG. 3 to FIG. 8 is adopted to perform the purification method of Example 1 as shown in FIG. 1. The specific parameters are as follows:
an inlet flow rate of the gas to be treated: 2.0 m/sec;
an inlet quantity of the gas to be treated: 162 m³/hr;
a length, a width and a height of the single treatment unit: 693 mm, 664 mm and 631 mm, respectively;
a length and a width of the windward panel formed with the inlet holes: 427 mm and 555 mm, respectively;
a quantity of the water mist continuously fed into the single treatment unit: 0.1 L/min;
a specific surface area of each porous substrate: 2800 m²/g;
a pore size of each porous substrate: 60 mesh;
a length, a width and a height of each porous substrate: 600 mm, 600 mm and 50 mm, respectively;
type of the porous substrate: foamed ceramic substrate;
an angle between two ultraviolet lamps: 30°;
a spacing between the ultraviolet lamps: 152.7 mm;
a wavelength of the ultraviolet lamps: about 365 nm; and
a power of the ultraviolet lamps: about 10 W.

During the purification process, a VOC contained in the AMC to be treated can be decomposed by the photocatalysts to form the first treated composition with a smaller molecular weight. Decomposition reactions of acetone with photocatalysts under light exposure are as follows.

$${CH}_{3} {COCH}_{3} + {O}_{2} {\to }^{hv} HCOOH + {CH}_{3} CHO$$

$${CH}_{3} CHO + \frac{1}{2} {O}_{2} {\to }^{hv} 2 HCOOH$$

$$2 HCOOH + {O}_{2} {\to }^{hv} 2 {CO}_{2} + 2 {H}_{2} O$$

Decomposition reaction of isopropanol with photocatalysts under light exposure is as follows.

$${C}_{3} {H}_{8} O + {O}_{2} {\to }^{hv} 3 {CO}_{2} + 4 {H}_{2} O$$

Decomposition reaction of toluene with photocatalysts under light exposure is as follows.

$${C}_{6} {H}_{5} C {H}_{3} + 9 {O}_{2} {\to }^{hv} 7 {CO}_{2} + 4 {H}_{2} O$$

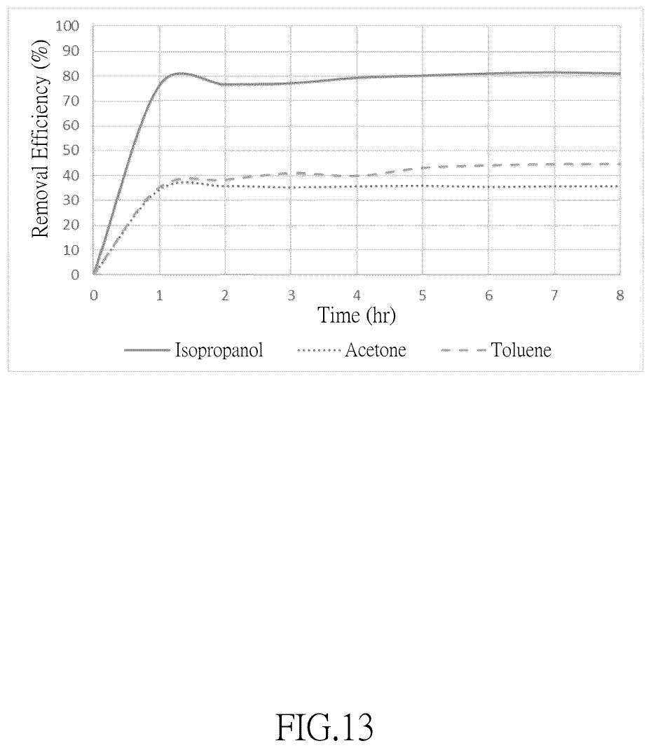

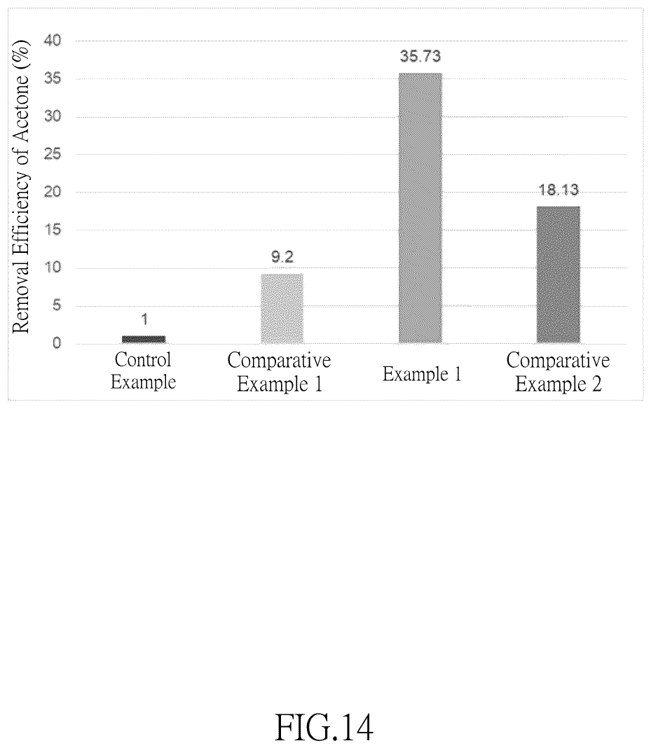

Ion chromatography (IC) is used to detect contents of isopropanol, acetone and toluene in both the AMC to be treated and the purified gas. The removal efficiency of each VOC is calculated by subtracting a percentage of a VOC content in the purified gas relative to its VOC content in the AMC to be treated from 100%. The results are shown in FIG. 13 and FIG. 14. FIG. 14 illustrates the removal efficiencies of acetone of Example 1, Comparative Examples 1 and 2, and Control Example after 1 hour of purification process. 2 This comparison is useful to evaluate the removal efficiency under the same purification time.

4 According to FIG. 13, the purification technology for the AMC is suitable to effectively remove the isopropanol, the acetone and the toluene from the AMC to be treated. The result of Example 1 shows that the purification method and purification system of the present invention exhibit excellent removal performance for these three VOCs. A high removal efficiency can be maintained even after prolonged purification, without needing to interrupt for photocatalysts regeneration.

### Control Example

Different from Example 1, Control Example refers to a system without any photocatalysts and the porous substrate. An equal quantity of water is introduced into the system, and the gas to be treated is fed into the system at the same inlet airflow rate and the inlet quantity as set in Example 1. The result is shown in FIG. 14.

### Comparative Example 1

Unlike Example 1 above, a system of Comparative Example 1 includes a porous substrate but does not include any photocatalysts. An equal quantity of water was introduced into the system, and the gas to be treated was fed into the system at the same inlet airflow rate and the inlet quantity as set in Example 1. The result is shown in FIG. 14.

### Comparative Example 2

Different from Example 1 above, a difference between Comparative Example 2 and Example 1 lies in that the water mist was not mixed with the AMC to be treated first, and the water mist was sprayed to the porous substrate after the reaction of the AMC to be treated with the photocatalysts. The inlet airflow rate and the inlet quantity of the AMC to be treated and the quantity of spraying the water mist were set basically the same as in Example 1. The result is shown in FIG. 14.

As shown in FIG. 14, in comparison with Control Example and Comparative Examples 1 and 2, Example 1 has the highest acetone removal efficiency, showing that an implementation of the purification method using the purification system for the AMC of the present invention indeed improves the VOCs removal efficiency.

### Comparative Example 3

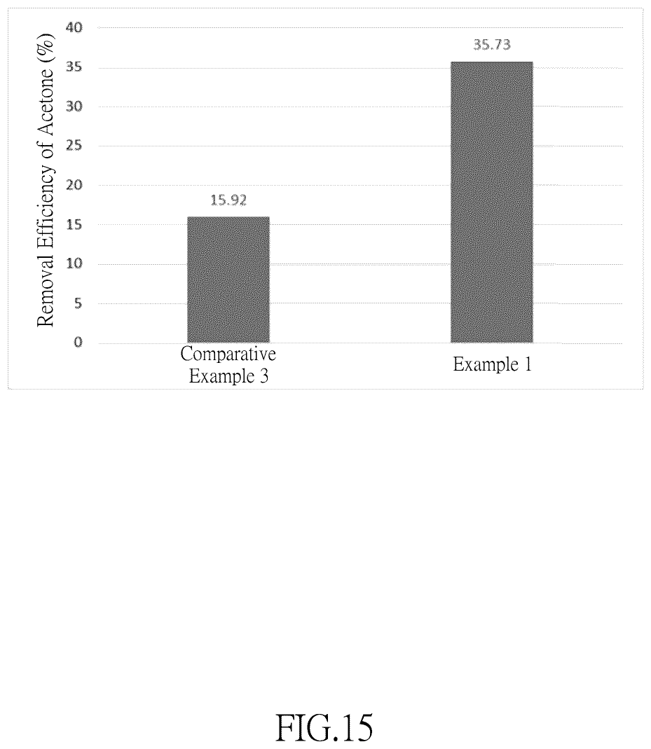

Unlike Example 1, the comparative example 3 adopts an existing device to purify the AMC, instead of the purification system of the present invention. The device adopted in Comparative Example 3 has photocatalysts, but does not comprise any porous substrates. During the purification process, the AMC to be treated is fed into a system with the same inlet airflow rate and the inlet quantity, but the AMC to be treated did not undergo a gas-liquid mixing with a liquid or a water mist before the photocatalysis. Specifically, the AMC to be treated is fed into the system to contact the photocatalysts, and then the same quantity of water as in Example 1 was introduced into the system. The result is shown in FIG. 15.

As shown in FIG. 15, Example 1 has the higher acetone removal efficiency than that of the Comparative Example 3, which shows that the implementing of the purification method with the present invention of the purification system for the AMC specifically improves the removal efficiency of the VOCs.

### Example 2

Different from Example 1 above, multiple treatment units are combined as needed. The purification system as shown in FIG. 8 and FIG. 9 are adopted for the purification. The specific parameters of Example 2 are as follows:
an inlet quantity of the gas to be treated: 2592 m³/hr;
a length, a width and a height of the single treatment unit: 693 mm, 664 mm and 631 mm, respectively;
a number of the treatment units of the first gas treatment module: 56; and
a quantity of the water mist continuously fed into the single treatment unit: 0.7 L/min.

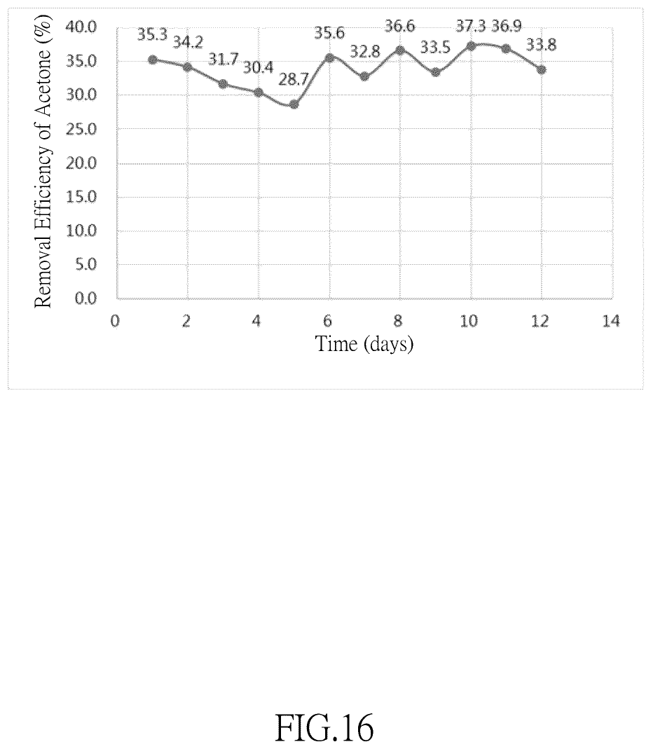

The results of purifying AMC by using the purification system of FIG. 8 and FIG. 9 are shown in FIG. 16. According to FIG. 16, even though a large amount of the AMC to be treated is purified, the acetone removal efficiency is still maintained at around 28% to 40%. The results show that the purification system for the AMC exhibit excellent purification performance, regardless of whether it processes a small or large amount of the AMC to be treated at the same time.

In summary, the present invention of the purification method and the purification system for the AMC are beneficial to shortening the treatment time required for AMC, increasing the purification rate for the AMC to be treated, prolonging the lifetime of photocatalysts, and solving the issue of frequent interruption during the purification process for photocatalyst regeneration. Moreover, the present invention enhances the overall purification performance and reduces the AMC content in the purified gas to a lower level. Therefore, the purification method and the purification system for the AMC of the present invention not only overcome the previous deficiencies but also contribute to achieving the ESG and SDGs goals.

What is claimed is:

1. A purification method for airborne molecular contaminants (AMC), the purification method comprising:
mixing an AMC to be treated and a first water mist to obtain a gas-liquid mixture;
subjecting the gas-liquid mixture to react with a photocatalyst on a porous substrate under a radiation source, so as to react the AMC to be treated into a first treated composition;
washing the first treated composition and the porous substrate with a second water mist to obtain a water washed solution, wherein the first treated composition is dissolved in the water washed solution; and
exhausting a purified gas and the water washed solution, wherein a molecular weight of the first treated composition is lower than a molecular weight of the AMC to be treated, and a content of the AMC in the purified gas is lower than a content of the AMC to be treated.

2. The purification method as claimed in claim 1, wherein the purification method comprises steps of:
feeding the AMC to be treated into a chamber by an inlet flow rate of 0.1 m/min to 2.5 m/min; and
mixing the AMC to be treated and the first water mist in the chamber, so as to obtain the gas-liquid mixture.

3. The purification method as claimed in claim 1, wherein an inlet quantity of the AMC to be treated is 20 m³/hr to 5000 m³/hr.

4. The purification method as claimed in claim 1, wherein the first water mist is equal to the second water mist, the AMC to be treated is mixed with the first water mist to obtain the gas-liquid mixture, and the first treated composition and the porous substrate are washed by the first water mist at a flow rate of 0.1 L/min to 1 L/min, so as to obtain the water washed solution.

5. The purification method as claimed in claim 1, wherein a specific surface area of the porous substrate is 1000 m²/g to 3500 m²/g.

6. The purification method as claimed in claim 1, wherein a pore size of the porous substrate is 50 mesh to 200 mesh.

7. The purification method as claimed in claim 1, wherein the radiation source is selected from the group consisting of an ultraviolet source, an infrared source, a microwave source, a plasma source, an X-ray source, an ion beam source, and any combinations thereof.

8. The purification method as claimed in claim 1, wherein the purification method comprises:
recycling the water washed solution;
separating the first treated composition from the water washed solution to obtain a regeneration solution;
spraying the regeneration solution to produce a regeneration water mist; and
mixing the regeneration water mist and the AMC to be treated to obtain the gas-liquid mixture, and washing the first treated composition and the porous substrate with a combination of the regeneration water mist and the second water mist.

9. A purification system for airborne molecular contaminants, the purification system comprising:
a chamber, having a channel formed with an air inlet and an air outlet connected to each other;
a first gas treatment module located between the air inlet and the air outlet, and the first gas treatment module including at least one treatment unit, each treatment unit including:
a housing having a windward panel formed with at least one inlet hole and a side panel formed with at least one outlet hole, the side panel positioned around the windward panel to form a treatment space;
at least one porous substrate with photocatalysts disposed in the treatment space; and
at least one radiation source facing to the at least one porous substrate with photocatalysts; and
a water mist production module including an inlet pipe and a water mist sprayer, the water mist sprayer installed on the windward panel, wherein the water mist sprayer includes a spray nozzle facing toward the treatment space.

10. The purification system as claimed in claim 9, wherein the windward panel has a bearing hole installed with the water mist sprayer, the number of the at least one inlet hole is plural, the inlet holes are formed around the bearing hole, and the number of the at least one outlet hole is plural, the inlet holes are formed along an airflow direction defined by the air inlet and the air outlet, the outlet holes are formed along a diffuser direction, and the diffuser direction is non-parallel to the airflow direction.

11. The purification system as claimed in claim 9, wherein the at least one porous substrate with photocatalysts comprises a first porous substrate with photocatalysts and a second porous substrate with photocatalysts located in the treatment space and opposite to each other, the number of the at least one radiation source is plural; parts of the radiation sources are spaced apart from each other and faced to the first porous substrate with photocatalysts, and other parts of the radiation sources are spaced apart from each other and faced to the second porous substrate with photocatalysts; wherein the first porous substrate with photocatalysts is positioned diagonally to the second porous substrate with photocatalysts, and a distance between one side of the first porous substrate with photocatalysts and one side of the second porous substrate with photocatalysts that are close to the windward panel is larger than a distance between the other side of the first porous substrate with photocatalysts and the other side of the second porous substrate with photocatalysts that are far from the windward panel.

12. The purification system as claimed in claim 11, wherein the radiation sources are multiple ultraviolet lamps, an angle between one of the ultraviolet lamps faced to the first porous substrate with photocatalysts and another one of the ultraviolet lamps faced to the second porous substrate with photocatalysts is 30° to 60°.

13. The purification system as claimed in claim 11, wherein the parts of the radiation sources are positioned between the first porous substrate with photocatalysts and the side panel, and the other parts of the radiation sources are positioned between the second porous substrate with photocatalysts and the side panel.

14. The purification system as claimed in claim 9, wherein a specific surface area of the at least one porous substrate is 1000 m²/g to 3500 m²/g.

15. The purification system as claimed in claim 9, wherein a pore size of the at least one porous substrate is 50 mesh to 200 mesh.

16. The purification system as claimed in claim 9, wherein a thickness of the at least one porous substrate is 10 mm to 100 mm.

17. The purification system as claimed in claim 9, wherein a material of the at least one porous substrate is selected from the group consisting of foamed ceramic, activated carbon, metal organic framework, foamed metal and any combinations thereof.

18. The purification system as claimed in claim 9, wherein the purification system comprises:
a water collection tank disposed under the first gas treatment module;
a return pipe connected with the water collection tank and the inlet pipe; and
a filtration unit installed on the return pipe and branched with a discharge pipe.

19. The purification system as claimed in claim 9, wherein the number of the at least one treatment unit is plural, and the treatment units are detachably assembled with each other to form the first gas treatment module.

20. The purification system as claimed in claim 19, wherein the treatment units of the first gas treatment module are arranged in a matrix, and the windward panels of the treatment units are arranged closely and extending to an inner wall of the channel.

21. The purification system as claimed in claim 9, wherein the purification system comprises a second gas treatment module, the first gas treatment module and the second gas treatment module are positioned between the air inlet and the air outlet and spaced from each other; the second gas treatment module comprises at least one treatment unit, the at least one treatment unit of the first gas treatment module is identical to the at least one treatment unit of the second gas treatment module.

22. The purification system as claimed in claim 21, wherein the second gas treatment module comprises multiple treatment units detachably assembled with each other, and the windward panels of the treatment units in the second gas treatment module are arranged closely and extending to an inner wall of the channel.
